(19) United States
(12) Patent Application Publication
(10) Pub. No.: US 2026/0290790 A1
(43) Pub. Date: Sep. 24, 2026

(54) STRUCTURAL BATTERY INTEGRATION FOR PROSTHETIC DEVICES

(71) Applicant: University of Central Florida Research Foundation, Inc., Orlando, FL (US)

(72) Inventors: Jayan Thomas, Orlando, FL (US); Leaford Henderson, Orlando, FL (US); Haryson Pierre Louis, Orlando, FL (US)

(73) Assignee: University of Central Florida Research Foundation, Inc., Orlando, FL (US)

(21) Appl. No.: 19/459,033

(22) Filed: Jan. 26, 2026

Related U.S. Application Data
(60) Provisional application No. 63/776,315, filed on Mar. 24, 2025.

(51) Int. Cl. H01M 4/36; A61F 2/50; A61F 2/54; A61F 2/60; H01M 4/02; H01M 4/04; H01M 4/38; H01M 4/50; H01M 4/583; H01M 4/62; H01M 50/403; H01M 50/434

(52) U.S. Cl. CPC H01M 4/364; A61F 2/54; A61F 2/60; H01M 4/0409; H01M 4/043; H01M 4/38; H01M 4/50; H01M 4/583; H01M 4/623; H01M 50/403; H01M 50/434; A61F 2002/5055; H01M 2004/027; H01M 2004/028; H01M 2220/30; H01M 2300/0011

(57) ABSTRACT

A prosthetic device includes at least one carbon fiber-based composite battery or energy storage device that forms part of an outer shell of the prosthetic device. The battery or energy storage device may include a first cathode with cathode material patches at spaced locations on a first carbon fiber mat and a first anode with anode material patches at spaced locations on a second carbon fiber mat. A ceramic separator is positioned between the first and second carbon fiber mats, and a filler material is provided in spaces between the cathode material patches and the anode material patches. The carbon fiber-based composite battery provides both mechanical structural support and electrical power for the prosthetic device, eliminating the need for separate external battery packs while reducing overall weight. A method of manufacturing the prosthetic device is also disclosed.

### CROSS-REFERENCE TO RELATED APPLICATIONS

This application claims the benefit of U.S. Provisional Patent Application No. 63/776,315, filed Mar. 24, 2025, the entire contents of which are hereby incorporated by reference in its entirety.

### RELATED PATENTS AND APPLICATIONS

The disclosure in this application relates to U.S. Pat. No. 11,686,011 issued Jun. 27, 2023 and entitled “Vertically-Aligned Graphene-Carbon Fiber Hybrid Electrodes And Methods for Making Same”; U.S. Pat. No. 12,094,654 and entitled “Storing Energy in Carbon Fiber-Based Electric Vehicle Body Panels”; U.S. patent application Ser. No. 17/842,145 filed Jun. 16, 2022 and entitled “Dual Function Energy-Storing Supercapacitor-Based Carbon Fiber Composite for Body Panels of A Vehicle”; International Patent Application No. PCT/US2022/033881 filed Jun. 16, 2022 and entitled “Dual Function Energy-Storing Supercapacitor-Based Carbon Fiber Composite for Body Panels of A Vehicle”; and U.S. patent application Ser. No. 18/776,924 filed Jul. 18, 2024 and entitled “Glass Fiber-Based Energy Storing Composites As High Strength Structural Panels”, the entire contents of all of which are hereby incorporated by reference in their entirety.

### STATEMENT REGARDING FEDERALLY SPONSORED RESEARCH OR DEVELOPMENT

This invention was made with Government support under Award #IIP 2122779, awarded by the National Science Foundation. The Government has certain rights in the invention.

### FIELD OF THE DISCLOSURE

The present disclosure relates generally to prosthetic devices, and more particularly to prosthetic devices integrated with structural carbon fiber-based batteries or energy storage devices.

### BACKGROUND

Prosthetic devices have evolved considerably over time, transitioning from early designs constructed from heavy materials such as wood and metal to modern implementations utilizing advanced composite materials. The introduction of carbon fiber composites has enabled the development of prosthetic limbs that are lighter and stronger than their predecessors, offering improved durability and flexibility for users.

Despite these advancements in structural materials, prosthetic devices that incorporate electronic components, motors, or sensors continue to face challenges related to power supply. Conventional prosthetic designs typically rely on external battery packs to provide electrical energy for these active components. Such battery packs add weight and bulk to the prosthetic device, which can affect user comfort and mobility, particularly during extended periods of use.

The additional weight from external battery systems can be especially burdensome for certain user populations, including children and elderly individuals, who may have reduced strength or endurance. Furthermore, the placement and size of conventional battery packs can affect the aesthetic appearance of prosthetic devices and may limit design options for prosthetic manufacturers.

Carbon fiber reinforced plastics and similar composite materials have demonstrated utility in various applications beyond prosthetics, including automotive body panels and sporting equipment. Research has explored the potential for integrating energy storage capabilities directly into carbon fiber composite structures, creating materials that can serve dual functions as both structural components and power sources.

The development of structural batteries and energy storage composites represents an area of ongoing interest in materials science. Such technologies aim to reduce the overall weight of devices by eliminating the distinction between structural components and energy storage systems. Water-based and non-toxic material formulations for such energy storage systems have also been explored to address safety and environmental considerations.

Accordingly, there remains interest in approaches that can address the challenges associated with powering prosthetic devices while maintaining or improving their weight, comfort, and functionality characteristics.

### SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In one aspect, a prosthetic device comprises at least one carbon fiber-based composite battery or energy storage device, wherein the at least one carbon fiber-based composite battery or energy storage device forms part of an outer shell of the prosthetic device. The prosthetic device may be a prosthetic limb, such as a prosthetic leg or arm.

In some aspects, the at least one battery or energy storage device comprises a first cathode including cathode material patches at spaced locations on a first carbon fiber mat, the deposited cathode material patches collectively having a first configuration; a first anode including anode material patches at spaced locations on a second carbon fiber mat, the deposited anode material patches collectively having a second configuration that matches the first configuration; and a first separator positioned between the first and second carbon fiber mats. The first and second carbon fiber mats may be stacked so that the cathode material patches substantially align with the anode material patches. The first separator may comprise a ceramic separator coating on each of the anode material patches and cathode material patches. The spaces between the cathode material patches and the anode material patches may include a filler material, such as an epoxy resin.

In some aspects, the anode material comprises Zinc, such as electrodeposited zinc nanosheets. In some aspects, the cathode material comprises MnO₂-multi-walled carbon nanotube nanocomposite. In some aspects, the cathode material comprises MnO₂ nanopowders, carbon black nanoparticles, single-walled carbon nanotubes, and polyvinylidene fluoride (PVDF). The first and second carbon fiber mats may comprise cross-woven carbon fiber mats. The ceramic separator may comprise molecular sieve powder and polyvinylidene fluoride (PVDF). The prosthetic device may further comprise an electrolyte comprising zinc sulfate and manganese sulfate.

In some aspects, the at least one carbon fiber-based composite battery or energy storage device weighs less than 15% of a conventional battery of equivalent capacity. The prosthetic device may further comprise a plurality of carbon fiber-based composite batteries connected in series. The at least one carbon fiber-based composite battery or energy storage device may provide both mechanical structural support and electrical power for the prosthetic device.

In another aspect, a method of manufacturing a prosthetic device comprises pretreating a first carbon fiber mat and a second carbon fiber mat to remove sizing; electrodepositing anode material onto the first carbon fiber mat to form an anode; depositing cathode material onto the second carbon fiber mat to form a cathode; applying a ceramic separator coating onto the anode and the cathode; stacking the first and second carbon fiber mats with the ceramic separator positioned therebetween; and forming the stacked first and second carbon fiber mats into an outer shell of the prosthetic device.

In some aspects, pretreating the first carbon fiber mat and the second carbon fiber mat comprises heating the carbon fiber mats with a flame to remove sizing and improve surface conductivity. Electrodepositing anode material may comprise electrodepositing zinc nanosheets onto the first carbon fiber mat in an electrolyte solution comprising zinc sulfate. Depositing cathode material may comprise preparing a slurry comprising MnO₂ nanopowders, carbon black nanoparticles, single-walled carbon nanotubes, and polyvinylidene fluoride (PVDF), and doctor blading the slurry onto the second carbon fiber mat. The method may further comprise cold pressing the deposited cathode material to improve surface uniformity. Applying the ceramic separator coating may comprise preparing a ceramic paste comprising molecular sieve powder and polyvinylidene fluoride (PVDF), and doctor blading the ceramic paste onto the anode and the cathode. The method may further comprise soaking the stacked first and second carbon fiber mats in an electrolyte solution comprising zinc sulfate and manganese sulfate. The method may further comprise applying an epoxy filler material to spaces between anode material patches and cathode material patches. The method may further comprise connecting a plurality of stacked carbon fiber mats in series to form a composite battery.

In yet another aspect, a carbon fiber-based structural battery for a prosthetic device comprises a first carbon fiber mat having anode material patches at spaced locations thereon; a second carbon fiber mat having cathode material patches at spaced locations thereon; a ceramic separator positioned between the first and second carbon fiber mats; and a filler material in spaces between the anode material patches and the cathode material patches. The structural battery is configured to form at least a portion of an outer shell of the prosthetic device and to provide both mechanical structural support and electrical power for the prosthetic device.

The foregoing general description of the illustrative embodiments and the following detailed description thereof are merely exemplary aspects of the teachings of this disclosure and are not restrictive.

### BRIEF DESCRIPTION OF THE DRAWINGS

A more complete understanding of the present disclosure, and the attendant advantages and features thereof, will be more readily understood by reference to the following description when considered in conjunction with the accompanying drawings wherein:

FIG. 1A shows a photograph of a Zn coated cross-woven carbon fiber mat (Zn-CF) anode;

FIG. 1B shows a photograph of a MnO₂ deposited cross-woven carbon fiber mat (MnO₂-CF) cathode;

FIG. 2A shows photographs of the Zinc anode (left) and the MnO₂ cathode (right) before deposition of a ceramic separator film;

FIG. 2B shows photographs of the Zinc anode (left) and the MnO₂ cathode (right) after deposition of the ceramic separator film;

FIG. 3 schematically shows a stacking pattern of the anode and cathode on cross-woven carbon fiber mats with separator films;

FIG. 4 schematically shows a pair of stacked anodes and cathodes on cross-woven carbon fiber mats with separator films connected in series to form a single composite battery;

FIG. 5A shows cyclic voltammetry (CV) curves at a scan rate of 0.1 mVs⁻¹ for a Zn—MnO₂ battery constructed on the cross-woven carbon fiber mat sheet;

FIG. 5B shows galvanostatic charge-discharge (GCD) cycling data at different cycling rates for a Zn—MnO₂ battery constructed on the cross-woven carbon fiber mat sheet; and

FIG. 6 schematically shows a prosthetic arm with an outer shell incorporating structural carbon fiber batteries.

### DETAILED DISCLOSURE

As required, embodiments are disclosed herein; however, it is to be understood that the disclosed embodiments are merely examples and that the methods described below can be embodied in various forms. Therefore, specific structural and functional details disclosed herein are not to be interpreted as limiting, but merely as a representative basis for teaching one skilled in the art to variously employ the present subject matter in virtually any appropriately detailed structure and function. Further, the terms and phrases used herein are not intended to be limiting, but rather, to provide an understandable description of the concepts.

It can be advantageous to set forth definitions of certain words and phrases used throughout this disclosure. The terms “a” or “an”, as used herein, are employed to describe elements and components described herein. This is done merely for convenience and to give a general sense of the scope of the disclosure. This description should be read to include one or at least one and the singular also includes the plural unless it is obvious that it is meant otherwise. The term plurality, as used herein, is defined as two or more than two. The term another, as used herein, is defined as at least a second or more.

The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, or C; A and B; A and C; B and C; and A, B, and C.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Note that not all of the activities described above in the general description or the examples are required, that a portion of a specific activity may not be required, and that one or more further activities can be performed in addition to those described. Still further, the order in which activities are listed is not necessarily the order in which they are performed.

In general, one aspect of the disclosure relates to eliminating the constraints external battery packs have on prosthetic devices by embedding energy storage directly into the prosthetic structure using carbon fiber-based materials. Current prosthetic designs are limited by bulky external battery sources, which add weight and reduce both efficiency and convenience for users, particularly those engaged in long-duration activities.

By leveraging advancements in materials science, the outer shell of prosthetic limbs can serve as both a load-bearing structure and a lightweight, efficient battery or energy storage unit. This dual-function design significantly reduces the weight of the prosthetic while also increasing its operational capacity, allowing users to go longer periods without the need for frequent recharging. The integrated energy storage solution provides a seamless and aesthetically pleasing alternative to conventional systems, addressing long-standing challenges in weight, space utilization, and power management in the field of prosthetics.

The dual-function outer shell represents a paradigm shift in how energy storage can be incorporated into wearable medical devices. Structural batteries or energy storage units embedded within carbon fiber composite materials offer users enhanced comfort, mobility, and functionality, revolutionizing the experience of wearing prosthetic limbs. The invention has far-reaching implications for the future of prosthetic design, improving the lives of users by offering a more integrated, efficient, and durable power solution.

With reference to the disclosures in the list of related patents and applications set forth above, the development of structural energy storage by integrating energy storage in carbon fiber reinforced plastics (CFRP) has been demonstrated. Building of this advance, the prosthetic limb's outer carbon fiber composite outer shell serves not only as a mechanical structure but also as an energy storage unit, effectively eliminating the need for a separate standalone heavy battery. These structural batteries (also called “weightless batteries”), weighing less than 15% of conventional batteries, are seamlessly embedded within the prosthetic's framework. This dual-function technology optimizes space, reduces weight, and significantly enhances mobility for users. Moreover, the use of non-toxic, water-based materials and advanced carbon fibers ensures that these prosthetics are environmentally sustainable and safe for long-term use. This advancement has the potential to revolutionize the quality of life for prosthetic users, offering a more streamlined, comfortable, and efficient alternative to traditional designs.

Unlike traditional designs in which batteries are separate units and therefore very heavy, the invention's integration of carbon fiber-based structural batteries into the prosthetic framework. allows the prosthetic limbs themselves to store energy, serving both as the device's structural support and power source. The structural batteries not only provide energy for motors and sensors but also contribute to the mechanical strength of the prosthetic limb. The structural batteries can be limited to using water and non-toxic materials. This dual functionality not only reduces the overall weight of the prosthetic but also optimizes space utilization, making the design more compact and user-friendly. Additionally, the use of advanced carbon fiber materials ensures that the prosthetics are durable and lightweight, contributing to improved mobility and comfort for the user.

An exemplary method for making a prosthetic device, integrated with structural carbon fiber batteries will now be described.

### Pretreatment of Carbon Fibers

If applicable, carbon fiber (CF) mats, which may be cross-woven carbon fiber mats (CWCFM), are first treated to remove the insulating polymer coating, or sizing, applied from the manufacturer to improve handling workability of the fiber sheets. Fiber sheets may be heated with a propane flame at a rate of, for example, ~1 in²/s to uniformly de-size the fibers. This approach rapidly improves the surface conductivity of the mats, while increasing the roughness, which improves adhesion of the active materials applied during the electrode fabrication.

### Exemplary Synthesis of Anode Materials and Anode Fabrication

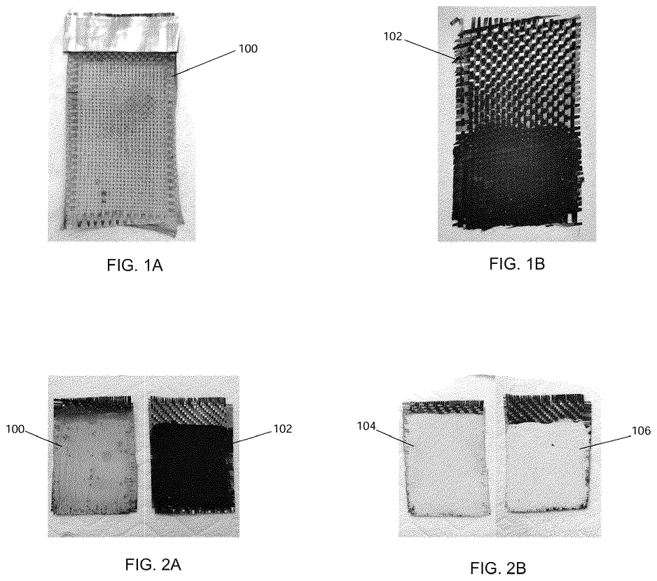

Zinc nanosheets are prepared directly on the pretreated CF sheets by, for example, electrodeposition in an electrolyte solution consisting of 1.0M zinc sulfate in deionized (DI) water, at a current density of 20 mAcm⁻² ordinarily for 20 minutes; the deposition time and current can be adjusted according to the desired areal capacity of the anode. FIG. 1A shows a Zn coated CWCFM (ZN-CF) anode 100. The invention contemplates the use of any suitable anodic material. For example, sodium titanium phosphate (NTP) or hard carbon microspheres could also be used as an anode material.

### Exemplary Synthesis of Cathode Materials and Cathode Fabrication

The cathode materials may be prepared through various processes.

In one approach, manganese oxide cathode materials may be prepared through a facile hydrothermal process. Potassium permanganate (KMnO₄; 1.317 g) and manganese sulfate monohydrate (MnSO₄·H₂O; 0.234 g) are dissolved in DI water with magnetic stirring for 15 minutes, before reacting at 160° C. in a Teflon® autoclave for 12 h. After cooling to room temperature, the MnO₂ precipitate is collected by filtration and washed 3× with DI water and then with ethanol, before drying at 80° C. for 12 h.

To prepare the MnO₂ cathodes on the CF sheets, a slurry is prepared by grinding together MnO₂ nanopowders, carbon black nanoparticles, single-walled carbon nanotubes (SWCNTs), and polyvinylidene fluoride (PVDF) in a ratio of 80:8:2:10 by weight with addition of N-methylpyrrolidone (NMP) to reach a uniform consistency. The slurry is then doctor bladed on the CF sheets followed by drying at 80° C. for 6 h and then cold pressing at 5 MPa for 1 minute to improve the surface uniformity of the cathode film. FIG. 1B shows a MnO₂ deposited CWCFM (MnO₂-CF) cathode 102. The invention contemplates the use of any suitable cathodic material. For example, sodium iron phosphate (NaFePO₄) or sodium manganese oxide (NaₓMnO₂) could also be used as a cathode material.

In another embodiment, synthesis of cathode materials and cathode fabrication of the Zn-ion battery is as follows.

Typical Zn—MnO₂ batteries employ hydrothermally prepared δ-phase MnO₂ powders with diameters of 1-5 microns across; in contrast, the following is a low temperature, non-hydrothermal synthetic approach to produce MnO₂ nanoplatelets, which allow for much closer packing between the MnO₂ active material and conductive carbon additives used in the slurry formation. In addition, the synthetic technique was further refined to produce a MnO₂-MWCNT nanocomposite material, which further enhances the charge transport of the active material. The combined effect of these modifications is to enhance the rate capacity of the Zn—MnO₂ battery, which would facilitate effective rapid charging of the prosthetic structural battery. This is important for the user functionality of the prosthetic devices, since typical slow charging would require the user to be without the use of the prosthetic arm for an extended period, and by enhancing the rate capacity of the cathode material, the charging time required for the structural battery has been reduced.

An example of this synthetic route for δ-phase MnO₂ is:
Acid-treated MWCNTs (100 mg) are sonicated in DI water (50 mL) for 15 minutes.
Once MWCNTs are fully dispersed, 2.45 g manganese acetate (Mn(OAc)₂) is dissolved with stirring at room temperature.
Potassium hydroxide (KOH) solution (2 g, dissolved in 50 mL DI water) is added dropwise to the manganese acetate/MWCNT dispersion at a rate of 1.5 mL s⁻¹.
Once the KOH solution is completely added, stirring is halted, and the solution is allowed to stand for 30 minutes.
After the 30 minutes standing period is complete, ammonium persulfate ((NH₄)₂S₂O₈) solution (2.05 g in 50 mL DI water) is added to the previous solution with stirring, over a period of 30 minutes.

The mixture is allowed to stand without stirring for 15 h, after which the solid MnO₂-MWCNT nanocomposite is collected by filtration and washing with DI water and ethanol, and dried at 80° C. for 12 h. After drying, coating on CF sheets is as described above.

### Exemplary Ceramic Separator (CS) and Electrolyte Preparation

(1) Electrolyte preparation: A 2M Zn²⁺/0.2M Mn²⁺ electrolyte solution is prepared by dissolving 5.75 g of zinc sulfate and 0.388 g of manganese sulfate in 10 mL of DI water while stirring.

(2) Ceramic power preparation and pretreatment: Solid beads of 13X molecular sieves (MS) are ground to a fine powder. 5 g of this powder is then soaked with 3 mL of the Zn²⁺/Mn²⁺ electrolyte solution for 24 h, after which the Zn²⁺/Mn²⁺ loaded powders are collected by filtering, washing to remove surface residues with DI water, and drying at 80° C. for 12 h.

(3) Ceramic separator paste preparation: pretreated MS powders (400 mg) are ground with PVDF (100 mg) for 10 minutes, before adding 4 mL NMP and grinding for a further 15 minutes to produce a translucent paste.

(4) Separator application and fabrication: A thin film of the ceramic paste is doctor-bladed on the surface of the anode and cathode before drying at 80° C. for 3 h. The process can be repeated to vary the final thickness of the separator film; and the thickness can be optimized by preparing a series of ceramic separator devices with varying thickness and studying the charge transfer kinetics using an electrochemical workstation. The film with optimized thickness is used for developing the carbon fiber zinc battery composite. The final device is prepared by coating a thin layer of paste on a single pre-coated cathode, before stacking it on top of a corresponding pre-coated anode and curing at 80° C. for 6 hours. This device has anode and cathode bonded together by the ceramic separator; the device is finally soaked for 24 hours in the electrolyte solution from step (1) before sealing for testing and use.

FIG. 2A shows photographs of the Zinc anode 100 (left) and the MnO₂ cathode 102 (right) before deposition of the ceramic separator film. FIG. 2B shows photographs of the Zinc anode 104 (left) and the MnO₂ cathode 106 (right) after deposition of the ceramic separator film.

### Device Assembly and Fabrication

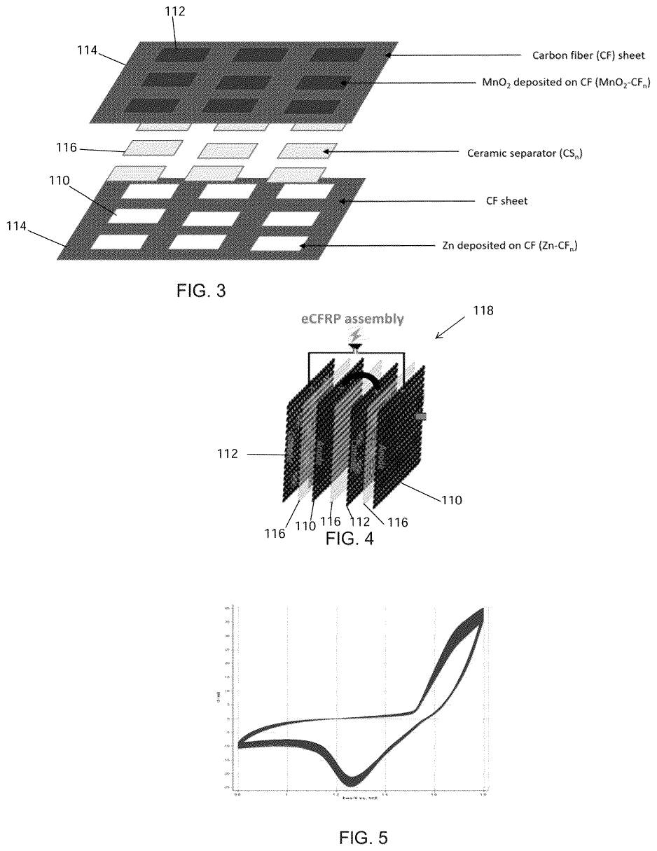

FIG. 3 shows an exemplary stacking pattern of the anode 110 and cathode 112 on CF mats 114 with separator films 116. FIG. 4 shows two pairs of anodes 110 and cathodes 112 on CF mats 114 with separator films 116 connected in series to fabricate a single composite battery (eCFRP) 118. In the configuration, CSₙ represents the ceramic separator between the anode (Zn-CF) and cathode (MnO₂-CF) of the n^{th} device. After preparing the dried CS film on both anode and cathode active areas, a final, thin coat of the CS paste would be applied to all the cathode surfaces of the upper device sheet, which will then be vertically stacked onto the anode surfaces on the lower device sheet. The stacked layers will then be cured to form a complete device with individually connected cells at each combination of Zn-CF |CS|(MnO₂-CF. The cured device stack will then be soaked for 24 hours in the electrolyte solution to infiltrate the individual cells, before removal. The remaining inactive areas on the CF mats will be applied with binding epoxy to form the bond with its adjacent CWCFMs. Epoxy will be applied on both sides to enable strong bonding between the layers of the adjacent devices.

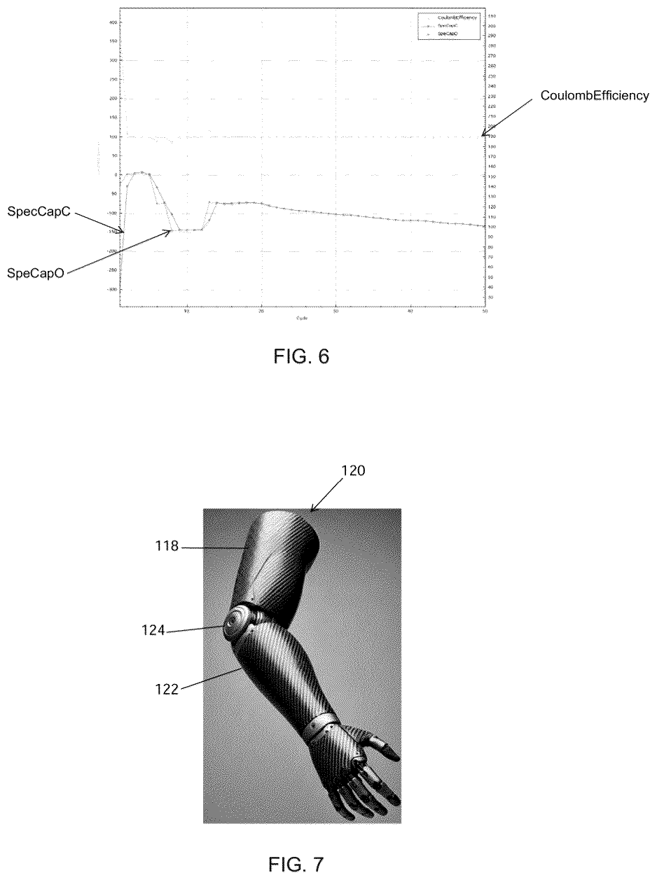

To get a required voltage and current, eCFRP structural prosthetic batteries are formed by layering anodes, cathodes, and separators soaked in electrolyte on a mold for getting the required prosthetic shape. As a non-limited exemplary embodiment, FIG. 6 shows eCFRP molded in the shape of an outer shell for an arm prosthetic device.

Specifically, a prosthetic device 120 is shown comprising at least one carbon fiber-based composite battery or energy storage device 118, wherein the at least one carbon fiber-based composite battery or energy storage device 118 forms part of an outer shell 122 of the prosthetic device 120. The prosthetic device 120 may be a prosthetic limb, such as a prosthetic leg or arm. The carbon fiber-based composite battery 118 may provide both mechanical structural support and electrical power for the prosthetic device 120, including power for electronic components 124 such as motors and sensors.

The prosthetic device 120 with the integral carbon fiber-based composite battery or energy storage device 118 may be formed using various molding techniques. In some aspects, the stacked carbon fiber mats with the anode, cathode, and separator layers may be placed on or within a mold having a shape corresponding to the desired prosthetic outer shell configuration. The mold may be a male mold, a female mold, or a matched mold set depending on the desired surface finish and dimensional tolerances.

In some aspects, the layered carbon fiber mats may be pre-impregnated with resin (prepreg) before being placed on the mold. In other aspects, a wet layup process may be used in which the carbon fiber mats are placed on the mold and resin is applied during the layup process. The epoxy filler material applied to the spaces between the anode material patches and the cathode material patches may also serve as a matrix material that bonds the layers together during curing.

The mold may be configured to apply pressure to the layered carbon fiber mats during curing to consolidate the layers and achieve the desired thickness and density. In some aspects, vacuum bagging may be used to apply uniform pressure to the layered structure during curing. In other aspects, an autoclave process may be used to apply both heat and pressure during curing.

The curing process may be performed at elevated temperatures to accelerate the curing of the epoxy or other matrix materials. In some aspects, the curing temperature may be selected to be compatible with the electrolyte and electrode materials to avoid degradation of the battery components.

After curing, the molded structural battery may be removed from the mold and trimmed or finished as needed to achieve the final prosthetic outer shell shape. The resulting outer shell may then be assembled with other prosthetic components to form the complete prosthetic device.

### Electrochemical Characterization: CV, GCD and EIS Result Discussion

The electrochemical performance of Zinc-ion batteries (ZIBs) was evaluated using a two-electrode setup with Zn/CF anode and MnO₂/CF cathode separated by an electrolyte infused solid ceramic separator, and a SP-150 electrochemical workstation at room temperature. The aqueous electrolyte for the electrochemical tests was a 2 M ZnSO₄+0.2 M MnSO₄ solution. Cyclic voltammetry (CV) tests were conducted over a voltage range of 0.8-1.8 V at a scan rate of 1.0m V s⁻¹, as shown in FIG. 5A.

During the cathodic scanning of MnO₂ in the ZIB, a single broad reduction peak at 1.25 V correspond to the reduction of Mn⁴⁺ to Mn³⁺. This reduction is identified as indicative of H⁺ and/or Zn²⁺ insertion/co-insertion. The discharge of MnO₂ results in the deposition of a zinc complex salt (ZnSO₄[Zn(OH)₂]₃·xH₂O) on the cathode surface due to local pH changes. Upon anodic scanning, the salt dissolves, and Mn³⁺ is re-oxidized to Mn⁴⁺. The Zn anode side undergoes Zn discharge as Zn→Zn²⁺+2^{e-}. The solid state ZIB device can be fabricated via leveraging the liquid electrolyte based ZIB chemistry. FIG. 5B shows evaluation of the electrochemical performance of the solid state ZIB device via charge discharge (GCD) data at different cycling rates.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended representative claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the representative claims.

After reading the disclosure, skilled artisans will appreciate that certain features are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated. It is intended that the following representative claims be interpreted to embrace all such variations and modifications.

What is claimed is:

1. A prosthetic device comprising at least one carbon fiber-based composite battery or energy storage device, wherein the at least one carbon fiber-based composite battery or energy storage device forms part of an outer shell of the prosthetic device.

2. The prosthetic device of claim 1, wherein the prosthetic device is a prosthetic limb.

3. The prosthetic device of claim 2, wherein the prosthetic limb is a prosthetic leg or arm.

4. The prosthetic device of claim 1, wherein the at least one battery or energy storage device comprises:
a first cathode including cathode material patches at spaced locations on a first carbon fiber mat, the deposited cathode material patches collectively having a first configuration;
a first anode including anode material patches at spaced locations on a second carbon fiber mat, the deposited anode material patches collectively having a second configuration that matches the first configuration; and
a first separator positioned between the first and second carbon fiber mats,
wherein the first and second carbon fiber mats are stacked so that the cathode material patches substantially align with the anode material patches;
wherein the first separator comprises a ceramic separator coating on each of the anode material patches and cathode material patches; and
wherein the spaces between the cathode material patches and the anode material patches include a filler material.

5. The prosthetic device of claim 4, wherein the filler material comprises an epoxy resin.

6. The prosthetic device of claim 4, wherein the anode material comprises Zinc.

7. The prosthetic device of claim 4, wherein the cathode material comprises MnO₂-multi-walled carbon nanotube nanocomposite.

8. The prosthetic device of claim 4, wherein the anode material comprises electrodeposited zinc nanosheets.

9. The prosthetic device of claim 4, wherein the first and second carbon fiber mats comprise cross-woven carbon fiber mats.

10. The prosthetic device of claim 4, wherein the ceramic separator comprises molecular sieve powder and polyvinylidene fluoride (PVDF).

11. The prosthetic device of claim 4, further comprising an electrolyte, wherein the electrolyte comprises zinc sulfate and manganese sulfate.

12. The prosthetic device of claim 1, further comprising a plurality of carbon fiber-based composite batteries connected in series.

13. The prosthetic device of claim 1, wherein the at least one carbon fiber-based composite battery or energy storage device provides both mechanical structural support and electrical power for the prosthetic device.

14. A method of manufacturing a prosthetic device, the method comprising:
pretreating a first carbon fiber mat and a second carbon fiber mat to remove sizing;
electrodepositing anode material onto the first carbon fiber mat to form an anode;
depositing cathode material onto the second carbon fiber mat to form a cathode;
applying a ceramic separator coating onto the anode and the cathode;
stacking the first and second carbon fiber mats with the ceramic separator positioned therebetween; and
forming the stacked first and second carbon fiber mats into an outer shell of the prosthetic device.

15. The method of claim 14, wherein electrodepositing anode material comprises electrodepositing zinc nanosheets onto the first carbon fiber mat in an electrolyte solution comprising zinc sulfate.

16. The method of claim 14, wherein depositing cathode material comprises:
preparing a slurry comprising MnO₂ nanopowders, carbon black nanoparticles, single-walled carbon nanotubes, and polyvinylidene fluoride (PVDF); and
doctor blading the slurry onto the second carbon fiber mat.

17. The method of claim 16, further comprising cold pressing the deposited cathode material to improve surface uniformity.

18. The method of claim 14, wherein applying the ceramic separator coating comprises:
preparing a ceramic paste comprising molecular sieve powder and polyvinylidene fluoride (PVDF); and
doctor blading the ceramic paste onto the anode and the cathode.

19. The method of claim 14, further comprising applying an epoxy filler material to spaces between anode material patches and cathode material patches.

20. A carbon fiber-based structural battery for a prosthetic device, the structural battery comprising:
a first carbon fiber mat having anode material patches at spaced locations thereon;
a second carbon fiber mat having cathode material patches at spaced locations thereon;
a ceramic separator positioned between the first and second carbon fiber mats; and
a filler material in spaces between the anode material patches and the cathode material patches,
wherein the structural battery is configured to form at least a portion of an outer shell of the prosthetic device and to provide both mechanical structural support and electrical power for the prosthetic device.
